SEQUENCE LISTING (13 sequences)

SEQ ID NO: 1 - RNA, 22 nt, Mus musculus
  source 1..22: mol_type = other RNA
  ncRNA 1..22: ncRNA_class = miRNA; note = microRNA miR-203b-3p
ttgaactgtcaagaaccactgg

SEQ ID NO: 2 - RNA, 22 nt, artificial sequence
  source 1..22: mol_type = other RNA
  misc_RNA 1..22: note = Antisense oligonucleotide of SEQ: ID: NO.1
ccagtggttcttgacagttcaa

SEQ ID NO: 3 - RNA, 23 nt, Homo sapiens
  source 1..23: mol_type = other RNA
  ncRNA 1..23: ncRNA_class = miRNA; note = microRNA miR-203b-3p
ttgaactgttaagaaccactgga

SEQ ID NO: 4 - RNA, 23 nt, artificial sequence
  source 1..23: mol_type = other RNA
  misc_RNA 1..23: note = Antisense oligonucleotide of SEQ: ID: NO.3
tccagtggttcttaacagttcaa

SEQ ID NO: 5 - RNA, 23 nt, artificial sequence
  source 1..23: mol_type = other RNA
  misc_RNA 1..23: note = Antisense oligonucleotide of SEQ: ID: NO.3
acaacttgttcttaacatgtcca

SEQ ID NO: 6 - RNA, 23 nt, artificial sequence
  source 1..23: mol_type = other RNA
  misc_RNA 1..23: note = Antisense oligonucleotide of SEQ: ID: NO.3
tcacgtacttcttaacatttaca

SEQ ID NO: 7 - RNA, 23 nt, artificial sequence
  source 1..23: mol_type = other RNA
  misc_RNA 1..23: note = Antisense oligonucleotide of SEQ: ID: NO.3
atgactggttcttaacagtagta

SEQ ID NO: 8 - RNA, 23 nt, artificial sequence
  source 1..23: mol_type = other RNA
  misc_RNA 1..23: note = Antisense oligonucleotide of SEQ: ID: NO.3
agctcaggttcttaacagttcaa

SEQ ID NO: 9 - RNA, 23 nt, artificial sequence
  source 1..23: mol_type = other RNA
  misc_RNA 1..23: note = Antisense oligonucleotide of SEQ: ID: NO.3
ttaagtggttcttaacagctaca

SEQ ID NO: 10 - RNA, 23 nt, artificial sequence
  source 1..23: mol_type = other RNA
  misc_RNA 1..23: note = Antisense oligonucleotide of SEQ: ID: NO.3
tgctgaggttcttaacagaccaa

SEQ ID NO: 11 - RNA, 23 nt, artificial sequence
  source 1..23: mol_type = other RNA
  misc_RNA 1..23: note = Antisense oligonucleotide of SEQ: ID: NO.3
tcaaacggttcttaacagttcaa

SEQ ID NO: 12 - RNA, 23 nt, artificial sequence
  source 1..23: mol_type = other RNA
  misc_RNA 1..23: note = Antisense oligonucleotide of SEQ: ID: NO.3
tcccgtggttcttaacagctcga

SEQ ID NO: 13 - RNA, 23 nt, artificial sequence
  source 1..23: mol_type = other RNA
  misc_RNA 1..23: note = Antisense oligonucleotide of SEQ: ID: NO.3
tgcagcggttcttaacagttaaa

(19) United States
(12) Patent Application Publication
(10) Pub. No.: US 2025/0043280 A1
(43) Pub. Date: Feb. 6, 2025

(54) ANTISENSE OLIGONUCLEOTIDE FOR USE IN THE TREATMENT OF PSORIASIS-INDUCED ITCHING AND PHOSPHOLIPID VESICLE COMPRISING SAID OLIGONUCLEOTIDE

(71) Applicant: FLONEXT S.R.L., Firenze (IT)

(72) Inventors: Romina NASSINI, Firenze (IT); Pierangelo GEPPETTI, Firenze (IT); Francesco DE LOGU, Castiglione della Pescaia (GR) (IT)

(73) Assignee: FLONEXT S.R.L., Firenze (IT)

(21) Appl. No.: 18/713,493

(22) PCT Filed: Nov. 23, 2022
(86) PCT No.: PCT/IB2022/061349

§ 371 Date: May 24, 2024

(30) Foreign Application Priority Data
Nov. 25, 2021 (IT) 102021000029894

(51) Int. Cl. C12N 15/113; A61K 9/00; A61K 9/06; A61K 9/127; A61P 17/06

(52) U.S. Cl. CPC C12N 15/113; A61K 9/0014; A61K 9/06; A61K 9/127; A61P 17/06; C12N 2310/113

(57) ABSTRACT

The present invention relates to the use of an oligonucleotide complementary to the sequence of human miR-203b-3p microRNA in the treatment of psoriasis-induced itching. The present invention concerns also a phospholipid vesicle comprising said oligonucleotide and the use of said phospholipid vesicle for the prevention and/or treatment of psoriasis-induced itching.

The present invention is related to the use of an oligonucleotide complementary to the sequence of human microRNA miR-203b-3p in the treatment of psoriasis-induced itching. The present invention refers also to a phospholipid vesicle comprising said oligonucleotide and the use of said phospholipid vesicle for the treatment of psoriasis-induced itching.

### STATE OF THE ART

Psoriasis is a chronic inflammatory skin disease that arises as a result of hyperproliferation of epidermal keratinocytes.

The most frequent form of psoriasis is the plaque psoriasis (about 80% of forms of psoriasis), characterized by the formation of bright red-colored plaques, covered with white scales that are, most frequently, but not exclusively, located on the extensor surfaces of the limbs. In addition to plaque psoriasis, there are other forms of psoriasis; pustular psoriasis, guttate psoriasis, inverse psoriasis and erythrodermic psoriasis.

Pustular psoriasis is distinguished from all other forms of psoriasis by the onset of yellowish pustules with purulent content. It is also associated with fever, malaise, paresthesias and burning sensations.

Guttate psoriasis is characterized by pink-red spots not covered with white scales.

As for inverse psoriasis, it causes large, pink erythematous lesions that, in the most severe cases, can be complicated by the formation of more or less deep fissures or rhagades that cause pain and can bleed.

The most severe form of psoriasis, fortunately rarer, is the erythrodermic psoriasis. Erythrodermic psoriasis is characterized by the presence of an erythema that affects more than 90% of the body surface, which is also associated with other important symptoms such as chills, cold sensation, itching and burning, general malaise, increased heart rate, asthenia, fever, swelling of lymph nodes and muscle and joint pains. This form of psoriasis is particularly disabling for the patient and in many cases requires hospitalization.

The Therapeutic Options Available Nowadays Aim to Prevent the Worsening of the Disease but not to Treat the Itching, a Characteristic Symptom of Psoriasis.

In particular, the therapeutic solutions offered consist of local treatments, ultraviolet (UV) light phototherapy, immunosuppressants and systemic treatments.

Specifically, local treatments are based on the application of corticosteroids, topical analogues of D3 vitamin, calcineurin inhibitors and/or tazarotene on the psoriatic lesion. However, the treatment with corticosteroids is not free of the common side effects caused by corticosteroids (irritation, burning, dry skin, hypopigmentation). Therefore, corticosteroids can only be used for short periods of time.

The treatment with analogues of calcineurin allows to avoid the side effects of corticosteroids but even today is not clear if they can increase the risk of lymphoma and skin cancer.

Tazarotene, a retinoid less effective than corticosteroids, can be used as an adjuvant and not in monotherapy.

Another option available for the treatment of psoriasis is the phototherapy (or UV light therapy), usually used in patients with diffuse psoriasis. Although the treatment is more controlled than topical therapy and can result in remissions over many months, repeated treatments can increase the incidence of UV-induced skin cancers and melanoma.

Immunosuppressants (e.g. hydroxyurea, 6-tyoguanine) cause very severe side effects, have narrow safety margins and are therefore reserved for cases of severe and resistant psoriasis.

Lastly, the systemic treatment can be carried out with retinoids (e.g. acitretin) or immunomodulatory agents [tumor necrosis factor-alpha (TNF-alpha) inhibitors such as etanercept, adalimumab, infliximab and golimumab]. However, due to the potential teratogenicity and long-term retention of retinoids in the body, women on acitretin therapy should not be pregnant and cannot have pregnancies for at least 2 years after the end of treatment.

Furthermore, with regard to the immunomodulatory agents mentioned above, their safety profile is still being studied. Indeed, as the TNF-alpha molecule is expressed in several organs and tissues, its inhibition could lead to severe side effects.

From a biological point of view, it would therefore be useful to identify a psoriasis-specific biomarker whose inhibition would not lead to heavy side effects.

On the one hand, the lack of side-effect-free drugs and, on the other hand, the known presence of symptoms that chronically afflict the patient with psoriasis, above all itching, prompt the search for new solutions for treating psoriasis.

There is therefore a clear need to provide a product that can prevent and/or treat the manifestations of psoriasis, particularly itching, without causing side effects.

### SUMMARY OF THE INVENTION

The Applicant addressed the problem of providing a new therapy for the treatment of psoriasis, and in particular psoriasis-induced itch that does not involve the drawbacks and side effects of current therapies.

The Applicant has surprisingly found that a new therapy based on the administration of an antisense oligonucleotide complementary to the miR-203b-3p microRNA sequence is useful specifically in treating psoriasis-induced itching.

In the present invention, the term “antisense oligonucleotides” refers to small, single-stranded DNA or RNA molecules complementary to a determined sequence. The sequence to which they bind is usually an mRNA molecule that, by binding to a specific nucleotide sequence, is no longer capable of being translated into the protein.

In particular, the antisense oligonucleotide for use according to the present invention is capable of binding the microRNA, miR-203b-3p, identified by the Applicant as being responsible for itching in psoriasis.

The binding of the antisense oligonucleotide according to the invention to the miR-203b-3p microRNA inhibits the activity of the miR-203b-3p microRNA and thus the itching.

In order to provide a valid therapy for the treatment of itching in psoriasis in humans, the Applicant has carried out studies on a murine model of psoriasis.

The miR-203b-3p microRNA present in the mouse has a sequence that differs from the human one by only one base (a cytosine in place of the fifth uracil ((SEQ: ID: NO. 1) shown in Example 2). The Applicant has found that application of imiquimod (5%) in mouse skin induced lesions similar to those observed in human psoriasis and, in particular, produced the thickening of the epidermis, skin redness and the presence of plaques, responses that are collectively quantified in the PASI (Psoriasis Area Severity Index) (FIG. 1B and Example 1). The treatment with imiquimod (5%) in mouse induced also itching.

The Applicant has found that this itching was inhibited by the subcutaneous administration of the oligonucleotide complementary to the murine miR-203b-3p microRNA sequence ((SEQ: ID: NO. 2) shown in Example 2).

As shown in the experimental section, by inhibiting the murine miR-203b-3p by means of an antisense oligonucleotide having a sequence entirely complementary (SEQ: ID: NO. 2) to the sequence of said murine miR-203b-3p microRNA it is possible to obtain the reduction of itching in psoriatic lesions induced by treatment with imiquimod (5%) in mouse.

This result paves the way for new therapies for the treatment of itching induced by psoriasis in humans. In particular, the Applicant has found that an antisense oligonucleotide complementary to the miR-203b-3p microRNA sequence present in humans (SEQ: ID: NO. 3) with a sequence (SEQ: ID: NO. 4) or sequence with identity percentage equal to at least 70% with respect to said (SEQ: ID: NO. 4), preferably equal to at least 75%, even more preferably 80%, is capable of alleviating or, depending on the severity of the lesion, completely treating psoriasis-induced itching. Accordingly, a first aspect of the present invention relates to an antisense oligonucleotide complementary to human miR-203b-3p microRNA (SEQ: ID: NO. 3), said oligonucleotide having sequence (SEQ: ID: NO. 4) or sequence with an identity percentage equal to at least 70% with respect to said sequence (SEQ: ID: NO. 4), preferably at least 75%, even more preferably at least 80% for use in the treatment of psoriasis-induced itching.

In a preferred embodiment, the oligonucleotide according to the invention has at least the 75% sequence identity with respect to said sequence (SEQ: ID: NO. 4).

In a particularly preferred embodiment, the oligonucleotide according to the invention has at least the 80% sequence identity with respect to said sequence (SEQ: ID: NO. 4).

The antisense oligonucleotide for use according to the invention comprises the central 8 nucleotide sequence 5′-UUCUUAAC-3′, which recognizes and inhibits the central portion 5′-GUUAAGAA-3′ of human miR-203b-3p. In fact, said central 8-nucleotide sequence 5′-UUCUUAAC-3′ is particularly relevant for pairing with the human miR-203b-3p and for its inhibition.

The antisense oligonucleotide described in the present patent application can be advantageously used for the treatment of itching induced by all the types of psoriasis, preferably for the treatment of itching induced by plaques psoriasis.

A second aspect of the present invention relates to a phospholipid vesicle that comprises at least one antisense oligonucleotide complementary to the human miR-203b-3p microRNA (SEQ: ID: NO. 3), said oligonucleotide having sequence (SEQ: ID: NO. 4) or sequence with an identity percentage equal to at least 70% with respect to said sequence (SEQ: ID: NO. 4), preferably at least 75%, more preferably at least 80%.

A third aspect of the present invention relates to a pharmaceutical composition comprising at least one phospholipid vesicle according to the second aspect of the invention.

### DETAILED DESCRIPTION OF THE INVENTION

The present invention relates to the use of an oligonucleotide complementary to the human miR-203b-3p microRNA sequence for the treatment of psoriasis-induced itching. In fact, it has been observed that miR-203b-3p is the main responsible for psoriasis-induced itching.

MicroRNAs are small endogenous molecules of single-stranded non-coding RNA approximately 20-22 nucleotides long and mainly active in the regulation of gene expression at transcriptional and post-transcriptional levels. In particular, microRNAs induce gene silencing by overlapping with complementary sequences present on messenger RNA (mRNA) molecules. Such binding results in the blocking of translation into protein of the mRNA target of the microRNA or degradation of said mRNA target of microRNA target.

MicroRNAs can act by cutting the mRNA molecule, destabilizing the mRNA molecule by shortening the poly(A) tail or decreasing the translation efficiency of the target mRNA. The Applicant has surprisingly discovered a new function of the miR-203b-3p microRNA. In fact, it has recently been observed that microRNAs not only have the function of post-transcriptional regulation of gene expression but also act as ligands, actively participating in the life of the cell.

The Applicant has advantageously conducted experiments on a murine model of psoriasis in order to assess the efficacy of an antisense oligonucleotide with respect to the murine miR-203b-3p microRNA in the treatment of psoriasis-induced itching (see experimental section). In particular, starting from the sequence of the murine miR-203b-3p microRNA (SEQ: ID: NO. 1), a sequence complementary to the murine miR-203b-3p (SEQ: ID: NO. 2) was obtained, whose efficacy in reducing itching in the murine model of psoriasis was observed.

The sequence of the murine miR-203b-3p microRNA (SEQ: ID: NO. 1) and the sequence of the oligonucleotide (SEQ: ID: NO. 2) complementary to the murine miR-203b-3p microRNA are indicated hereinafter:

Sequence of the Murine miR-203b-3p microRNA (SEQ: ID: NO. 1)

5′-UUG AAC UGU CAA GAA CCA CUG G-3′

Sequence of the oligonucleotide complementary to murine miR-203b-3p microRNA (SEQ: ID: NO. 2)

5′-CCA GUG GUU CUU GAC AGU UCA A-3′

The Applicant has surprisingly found that a new therapy based on the administration of an antisense oligonucleotide complementary to the human miR-203b-3p microRNA sequence is useful specifically in treating the psoriasis-induced itching in subjects suffering from psoriasis.

Accordingly, a first aspect of the present invention relates to an antisense oligonucleotide complementary to the human miR-203b-3p microRNA (SEQ: ID: NO. 3), said oligonucleotide having sequence (SEQ: ID: NO. 4) or sequence having an identity percentage equal to at least 70% with respect to said sequence (SEQ: ID: NO. 4), preferably at least 75%, more preferably at least 80%, for use in the treatment of psoriasis-induced itching.

In the present invention, the term “identity” means sequence identity, i.e. the degree of exact correspondence between two aligned oligonucleotide sequences (also taking GAP into account).

The antisense oligonucleotide sequence for use according to the invention is shown hereinafter and indicated with the identifier (SEQ: ID: NO. 4).

Sequence of the Antisense Oligonucleotide (SEQ: ID: NO. 4) Complementary to the Human miR-203b-3p microRNA

(SEQ: ID: NO. 4)
5′-UCC AGU GGU UCU UAA CAG UUC AA-3′

The antisense oligonucleotide for use according to the invention is capable of pairing with the human miR-203b-3p microRNA and of inhibiting the action thereof.

In particular, the human miR-203b-3p microRNA is a microRNA having the sequence shown hereinafter and indicated in the present invention with the identifier (SEQ: ID: NO. 3).

Sequence of the Human miR-203b-3p microRNA-(SEQ: ID: NO. 3)

(SEQ: ID: NO. 3)
5′-UUG AAC UGU UAA GAA CCA CUG GA-3′.

The miR-203b-3p is a microRNA highly expressed at skin level, and results linked to the mechanisms of skin morphogenesis. In fact, it has been shown that in embryonic stages this microRNA is highly expressed in cells of the supra-basal layer of the epidermis and is linked to the regulation of proliferation and differentiation mechanisms of epidermal cells (Sand et al, MicroRNAs and the skin: tiny players in the body's largest organ, J Dermatol Sci, 2009 53:169-75;).

The antisense oligonucleotide for use according to the invention is preferably constituted by 23 nucleotides and has a sequence entirely complementary to the sequence of the human miR-203b-3p microRNA (SEQ: ID: NO. 4) or a sequence with an identity percentage equal to at least 70% with respect to said sequence (SEQ: ID: NO. 4), preferably at least 75%, more preferably at least 80%.

The (SEQ: ID: NO. 4) is a sequence entirely complementary to the human miR-203b-3p microRNA sequence (SEQ: ID: NO. 3).

In a particularly preferred embodiment, said antisense oligonucleotide comprises the central 8-nucleotide sequence 5′-UUCUUAAC-3′ complementary to the central portion of the human miR-203b-3p microRNA having sequence 5′-GUUAAGAA-3′. In particular, the first uracil (U) of the central sequence UUCUUAAC corresponds to the base of the ninth nucleotide and the last cytosine (C) of the central sequence corresponds to the base of the sixteenth nucleotide in the oligonucleotide for use according to the invention. Thus, the central sequence runs from the ninth nucleotide to the sixteenth nucleotide in the oligonucleotide for use according to the invention of 23 nucleotides.

The (SEQ: ID: NO. 4) comprises a central sequence of 8 nucleotides 5′-UUCUUAAC-3′, named CORE sequence inhibitor. Said central sequence or CORE sequence inhibitor is the central sequence of the (SEQ: ID: NO. 4) of the oligonucleotide for use according to the invention, above underlined and in bold.

The central sequence, or CORE sequence inhibitor, is capable of pairing with and inhibiting the central portion of the human miR-203b-3p microRNA. The central portion of the human miR-203b-3p microRNA is the CORE sequence of the human miR-203b-3p microRNA and has sequence 5′-GUUAAGAA-3′.

The central portion of the human miR-203b-3p microRNA is underlined and in bold within the entire human miR-203b-3p microRNA sequence (SEQ: ID: NO. 3).

(SEQ: ID: NO. 3)
5′-UUG AAC UGU UAA GAA CCA CUG GA-3′.

According to the invention, the antisense oligonucleotide having sequence with an identity percentage equal to at least 70% with respect to the sequence (SEQ: ID: NO. 4), preferably at least 75%, more preferably at least 80%, is capable of reducing the psoriasis-induced itching. It is essential that the antisense oligonucleotide for use according to the invention recognizes the central portion of the human miR-203b-3p microRNA.

Therefore, the oligonucleotide for use according to the invention having sequence with an identity percentage equal to at least 70% with respect to the sequence (SEQ: ID: NO. 4), preferably at least 75%, more preferably at least 80% comprising the central sequence 5′-UUCUUAAC-3′ is capable of reducing the psoriasis-induced itching.

The antisense oligonucleotide with an identity percentage equal to at least 70% with respect to the oligonucleotide for use according to the invention (SEQ: ID: NO. 4) may have a sequence selected from the following sequences:

(SEQ: ID: NO. 5)
5′-ACA ACU UGU UCU UAA CAU GUC CA-3′
(SEQ: ID: NO. 6)
5′-UCA CGU ACU UCU UAA CAU UUA CA-3′
(SEQ: ID: NO. 7)
5′-AUG ACU GGU UCU UAA CAG UAG UA-3′
(SEQ: ID: NO. 8)
5′-AGC UCA GGU UCU UAA CAG UUC AA-3′
(SEQ: ID: NO. 9)
5′-UUA AGU GGU UCU UAA CAG CUA CA-3′
(SEQ: ID: NO. 10)
5′-UGC UGA GGU UCU UAA CAG ACC AA-3′
(SEQ: ID: NO. 11)
5′-UCA AAC GGU UCU UAA CAG UUC AA-3′
(SEQ: ID: NO. 12)
5′-UCC CGU GGU UCU UAA CAG CUC GA-3′
(SEQ: ID: NO. 13)
5′-UGC AGC GGU UCU UAA CAG UUA AA-3′

The sequences (SEQ: ID: NO. 5)-(SEQ: ID: NO. 7) have a variation of 7 bases with respect to the antisense oligonucleotide sequence (SEQ: ID: NO. 4) completely complementary to the human miR-203b-3p microRNA (SEQ: ID: NO. 3).

In the present invention, a sequence having a variation of 7 bases with respect to the sequence (SEQ: ID: NO. 4) is intended to have a sequence identity of 70% with respect to said sequence (SEQ: ID: NO. 4).

The sequences (SEQ: ID: NO. 8)-(SEQ: ID: NO. 10) have a variation of 5 bases with respect to the antisense oligonucleotide sequence (SEQ: ID: NO. 4) completely complementary to the human miR-203b-3p microRNA (SEQ: ID: NO. 3).

In the present invention, a sequence having a variation of 5 bases with respect to the sequence (SEQ: ID: NO. 4) is intended to have a sequence identity of 78% with respect to said sequence (SEQ: ID: NO. 4).

The sequences (SEQ: ID: NO. 11)-(SEQ: ID: NO. 13) have a variation of 3 bases with respect to the antisense oligonucleotide sequence (SEQ: ID: NO. 4) completely complementary to the human miR-203b-3p microRNA (SEQ: ID: NO. 3). In the present invention, a sequence having a variation of 3 bases with respect to said sequence (SEQ: ID: NO. 4) is intended to have a sequence identity of 87% with respect to said sequence (SEQ: ID: NO. 4).

In an embodiment, the antisense oligonucleotide may have a sequence having a variation of 1 or 2 bases with respect to the sequence of the antisense oligonucleotide (SEQ: ID; NO. 4).

In the present invention, a sequence having a variation of 1 or 2 bases with respect to the sequence (SEQ: ID: NO. 4) is intended to have a sequence identity higher than 90% with respect to said sequence (SEQ: ID. NO. 4).

Furthermore, said sequences (SEQ: ID: NO. 5)-(SEQ: ID: NO. 13) comprise all of them the central sequence 5′-UUCUUAAC-3′ as defined in the present invention, underlined within the sequences.

The antisense oligonucleotide for use according to the invention is capable of recognizing and binding the miR-203b-3p but is not capable of recognizing and binding the miR-203a-3p.

In a particularly preferred embodiment, the oligonucleotide for use according to the invention is an antagomir of the human miR-203b-3p microRNA.

In biotechnologies, the term “antagomir” is intended to indicate a class of chemically engineered oligonucleotides used to silence endogenous microRNAs of interest. In particular, an antagomir is a synthetic RNA perfectly complementary to the microRNA of interest, i.e. the microRNA that is intended to inhibit. The structure of antagomirs has modifications that make them more resistant to degradation. Such modifications may, for example, be modifications of the nucleotide sugar, of the nucleobase or of internucleotide linkages.

In particular, the use according to the invention relates to the treatment of itching induced by all types of psoriasis: plaque psoriasis, pustular psoriasis, guttate psoriasis, inverse psoriasis and erythrodermic psoriasis. Preferably, the antisense oligonucleotide complementary to the human miR-203b-3p microRNA (SEQ: ID: NO. 3), said oligonucleotide having sequence (SEQ: ID: NO. 4) or sequence having an identity percentage of at least 70% with respect to said (SEQ: ID: NO. 4) can be used for the treatment of itching induced by plaque psoriasis.

By pairing with the human miR-203b-3p microRNA (SEQ: ID: NO. 3), the oligonucleotide for use according to the invention is capable of inhibiting the direct activity of the human miR-203b-3p microRNA on the mechanism responsible for psoriasis-induced itching.

A second aspect of the present invention is a product comprising an antisense oligonucleotide complementary to the human miR-203b-3p microRNA. In particular, the second aspect of the present invention is a phospholipid vesicle that comprises at least one antisense oligonucleotide complementary to the human miR-203b-3p microRNA (SEQ: ID: NO. 3), said oligonucleotide having sequence (SEQ: ID: NO. 4) or sequence with an identity percentage equal to at least 70% with respect to said sequence (SEQ; ID: NO. 4), preferably at least 75%, more preferably at least 80%.

As above described, (SEQ: ID: NO. 4) is the sequence entirely complementary to the human miR-203b-3p microRNA (SEQ: ID: NO. 3).

The phospholipid vesicle according to the invention may comprise one or more of the oligonucleotides having sequence entirely complementary to the human miR-203b-3p microRNA (SEQ: ID: NO. 4) or sequence with at least 70% with respect to the sequence (SEQ: ID: NO. 4), preferably at least 75%, more preferably at least 80%.

In an embodiment, the vesicle according to the invention may comprise one or more of the oligonucleotides having sequence (SEQ: ID: NO. 5), (SEQ: ID: NO. 6) and (SEQ: ID: NO. 7).

In an embodiment, the vesicle according to the invention may comprise one or more of the oligonucleotides having sequence (SEQ: ID: NO. 8), (SEQ: ID: NO. 9) and (SEQ: ID: NO. 10).

In an embodiment, the vesicle according to the invention may comprise one or more of the oligonucleotides having sequence (SEQ: ID: NO. 11), (SEQ: ID: NO. 12) and (SEQ: ID: NO. 13).

In another embodiment, the phospholipid vesicle may comprise one or more of the oligonucleotides having sequence (SEQ: ID: NO. 5), (SEQ: ID: NO. 6) and (SEQ: ID: NO. 7) and/or one or more of the oligonucleotides having sequence (SEQ: ID: NO. 8), (SEQ: ID: NO. 9) and (SEQ: ID: NO. 10) and/or one or more of the oligonucleotides having sequence (SEQ: ID: NO. 11), (SEQ: ID: NO. 12) and (SEQ: ID: NO. 13).

In a further embodiment, the phospholipid vesicle may comprise the antisense oligonucleotide (SEQ: ID: NO. 4) and/or one or more of the oligonucleotides having sequence (SEQ: ID: NO. 5), (SEQ: ID: NO. 6) and (SEQ: ID: NO. 7) and/or one or more of the oligonucleotides having sequence (SEQ: ID: NO. 8), (SEQ: ID: NO. 9) and (SEQ: ID: NO. 10) and/or one or more of the oligonucleotides having sequence (SEQ: ID: NO. 11), (SEQ: ID: NO. 12) and (SEQ: ID: NO. 13).

The phospholipid vesicle according to the invention can be selected from liposomes, phytosomes, micelles, or mixtures thereof, preferably from liposomes.

Liposomes are a widely exploited delivery system for the delivery of poorly absorbable or poorly soluble actives, including pharmacological actives.

Due to the tenside nature of phospholipids that constitute them, liposomes “encapsulate” the active substance, in the present case one or more oligonucleotides for use according to the invention.

Advantageously, the constituents of the phospholipid layers of the liposome are biocompatible and, therefore, the liposome does not cause adverse effects.

Thus, it is clear that the product according to the invention advantageously allows to deliver high concentrations of the antisense oligonucleotide as above described, so that it can act on the target microRNA, i.e. the human miR-203b-3p microRNA, and contribute to the reduction of psoriasis-induced itching, if not to its complete elimination.

In an embodiment, the phospholipid vesicle according to the invention is a phytosome.

Phytosomes are release systems structurally related to liposomes prepared by attaching individual ingredients of herbal extracts to phosphatidylcholine.

Phytosomes are structures wherein the active ingredient is anchored to the polar head of the phospholipid and becomes integral part of the vesicular membrane, unlike liposomes, wherein the active ingredient is generally contained within the vesicular structure formed by phospholipids.

In another embodiment, the phospholipid vesicle according to the invention is a micelle. Micelles are spherical particles, wherein phospholipids are arranged with the polar heads on the outside, towards the aqueous environment, and the tails facing the inside of micelles. According to said embodiment, micelles can be single-layer micelles (monolayer) or bilayer micelles (bilayer).

In a particularly preferred embodiment, said phospholipid vesicle is a liposome.

In particular, the liposome according to the preferred embodiment of the present invention has dimensions between 100 nm and 800 nm, preferably between 200 nm and 600 nm, even more preferably 400 nm.

Furthermore, said liposome is preferably characterized by a polydispersity index of between 0.2 and 0.4, preferably of 0.3.

The polydispersity index is a measure of the heterogeneity of a sample based on size. Polydispersion can occur due to size distribution in a sample or agglomeration or aggregation of the sample during insulation or analysis.

The phospholipid vesicle is preferably constituted by corn lecithin, sunflower lecithin, soy lecithin, more preferably by corn lecithin.

The phospholipid vesicle according to the present invention, preferably a liposome, can be used for the treatment of itching induced by plaque psoriasis, pustular psoriasis, guttate psoriasis, inverse psoriasis and erythrodermic psoriasis, preferably plaque psoriasis. In particular, said phospholipid vesicle can be used for topical use on psoriatic lesions in order to achieve a reduction in itching or a complete elimination thereof.

A third aspect of the present invention relates to a pharmaceutical composition comprising one or more phospholipid vesicles according to the second aspect of the invention and as described above.

In an embodiment, the pharmaceutical composition according to the invention may comprise a mixture of liposomes, phytosomes, and micelles comprising one or more oligonucleotides for use according to the invention.

In an embodiment, the composition may comprise at least one phospholipid vesicle comprising at least one antisense oligonucleotide having sequence (SEQ: ID: NO. 4) and/or sequence with and identity percentage equal to at least 70% with respect to sequence (SEQ: ID: NO. 4), preferably at least 75%, more preferably at least 80%. According to the invention, the composition may further comprise one or more pharmaceutically suitable or pharmaceutically acceptable excipients.

The expression “pharmaceutically acceptable” is intended to define, without any particular limitation, any material suitable for the preparation of a pharmaceutical composition to be administered to a living being.

The expression “pharmaceutically suitable excipients” is intended to mean all solvents, diluents or other vehicles, dispersions or suspension aids, surfactants, isotonic agents, thickeners or emulsifying agents, preservatives, solid binders, lubricants and the like.

Some examples of materials that may serve as pharmaceutically acceptable excipients include, but are not limited thereto, sugars such as lactose, glucose and sucrose; starches such as corn starch; cellulose and its derivatives, such as sodium carboxymethyl cellulose, ethyl cellulose and cellulose acetate, powdered tragacanth gum, malt, gelatin, talc, excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate, agar, buffering agents such as magnesium hydroxide and aluminium hydroxide, alginic acid, apyrogenic water, isotonic salt solution, ethyl alcohol and phosphate buffer solutions, other compatible non-toxic lubricants such as sodium lauryl sulphate and magnesium stearate, coloring agents, releasing agents, coating agents, sweetening agents, flavoring and perfuming agents, preservatives and antioxidants.

Pharmaceutical forms may also contain other traditional ingredients such as preservatives, stabilizers, surfactants, buffers, salts for osmotic pressure control, emulsifiers, sweeteners, coloring agents, flavourings and similar.

The pharmaceutical composition according to the third aspect of the invention may be formulated for topical administration, for example in the form of a cream, powder or ointment. In another embodiment, the pharmaceutical composition has formulation useful for application in the form of a medicated patch. Medicated patches are products for dermal use that are applied to the skin and release defined amounts of drug in a controlled and constant manner.

In a preferred embodiment, the composition of the invention is formulated in the form of a cream.

Such a composition, preferably in the form of a cream, can be applied topically on one or more psoriatic lesions to achieve a gradual reduction of itching or a complete healing therefrom. Therefore, the composition according to the invention can be used for the topical treatment of psoriasis-induced itching, in particular plaque psoriasis, pustular psoriasis, guttate psoriasis, inverse psoriasis and erythrodermic psoriasis.

The present description further relates to a method of treatment of psoriasis-induced itching that comprises the topical application of at least one antisense oligonucleotide having sequence (SEQ: ID: NO. 4) and/or sequence having an identity percentage equal to at least 70% with respect to the sequence (SEQ: ID: NO. 4), preferably at least 75%, more preferably at least 80%.

In particular, said method of treatment of psoriasis-induced itching comprises the topical application of one or more phospholipid vesicles as above described, preferably in the form of a cream, on at least one psoriatic lesion of a subject suffering from psoriasis.

In an embodiment, the treatment method may also comprise the administration of a drug commonly used for the treatment of psoriasis.

Examples of such drugs are corticosteroids, topical analogues of D3 vitamin, calcineurin inhibitors and/or tazarotene.

One or more of the above-mentioned drugs may be administered together with at least one oligonucleotide having sequence (SEQ: ID: NO. 4) and/or sequence with an identity percentage equal to at least 70%, preferably at least 75%, more preferably at least 80%, with respect to the sequence (SEQ: ID: NO. 4).

One or more of the drugs conventionally used for the treatment of psoriasis, preferably selected from corticosteroids, topical analogues of D3 vitamin, calcineurin inhibitors and/or tazarotene, may be administered before, after, or concurrently with the topical treatment with the antisense oligonucleotide as above defined, possibly encapsulated in a phospholipid vesicle as above defined, or with the pharmaceutical composition according to the third aspect of the invention.

Thus, a combination of a phospholipid vesicle according to the second aspect of the invention with corticosteroids, topical analogues of D3 vitamin, calcineurin inhibitors and/or tazarotene for simultaneous, separate or sequential use in the treatment of psoriasis-induced itching may be particularly effective in determining the complete healing of the psoriatic lesion of interest.

### BRIEF DESCRIPTION OF THE FIGURES

FIGS. 1 and 2 show experiments performed on a murine model of psoriasis.

FIG. 3 shows data obtained in experiments on psoriasis model mice and control mice that were administered an oligonucleotide having a sequence entirely complementary to the central portion of the murine miR-203b-3p microRNA.

A detailed description of the figures can be found in the examples hereinafter.

### EXAMPLES

### Example 1-Murine Model of Psoriasis

The study of the inhibition of murine miR-203b-3p microRNA by an antisense oligonucleotide having a sequence entirely complementary to murine miR-203b-3p microRNA was conducted on a murine model of psoriasis induced by local (skin) treatment with imiquimod (5%) for 6 consecutive days.

The in vivo experiments were performed in compliance with the Italian legislation (DLgs 26/2014) and the guidelines provided by the European regulation (EU Directive 2010/63/EU). The study was conducted after the approval of the protocol by the Ministry of Health (research permit #511/2021-PR).

Male C57BL/6J mice 5-8 weeks old and weighing 22-25 g supplied by the company Charles River (Milan, Italy) were used for the experiments. A total of 24 mice were used to perform the experiments hereinafter described. The animals were kept in a controlled temperature and humidity environment (12-hour dark/light cycle, free access to food and water). The experiments were performed in a temperature-controlled room (20 to 22° C.) between 8 am and 8 pm. At the end of the experiment, the animals were euthanized by inhalation of an O₂ 50%/CO₂ 50% mixture for 1 minute.

The day before the experiment the mice (24) were shaved along their back using an electric razor, the remaining hair was removed with a depilatory cream.

The following day the mice (n=12) have received a topical application of 62.5 mg of imiquimod cream (5%, Aldara; 3M Pharmaceuticals, UK) on the shaved portion, corresponding to a daily dose of 3.125 mg of active compound. The dose used is the one which results to cause a reproducible skin inflammation with characteristics (specific cytokine expression patterns, histopathological changes and cellular infiltrates) similar to those seen in psoriatic patients (Van der Fits et al., 2009 Imiquimod-Induced Psoriasis-Like Skin Inflammation in Mice Is Mediated via the IL-23/IL-17 Axis J Immunol, 2009, 182 (9) 5836-5845.; Yoshiki, R. et al. 2014 IL-23 from Langerhans Cells Is Required for the Development of Imiquimod-Induced Psoriasis-Like Dermatitis by Induction of IL-17A-Producing yδ T Cells. J. Invest. Dermatol. 134. 1912-21).

Control animals (n=12) were treated with the imiquimod vehicle that consists of vaseline cream (control cream).

The application of the cream was performed by two operators, one of whom held the animal still and the other provided for the application of the cream using a cotton swab. The imiquimod cream (5%) or the control cream were applied on the shaved skin of the mice back every day for 6 consecutive days.

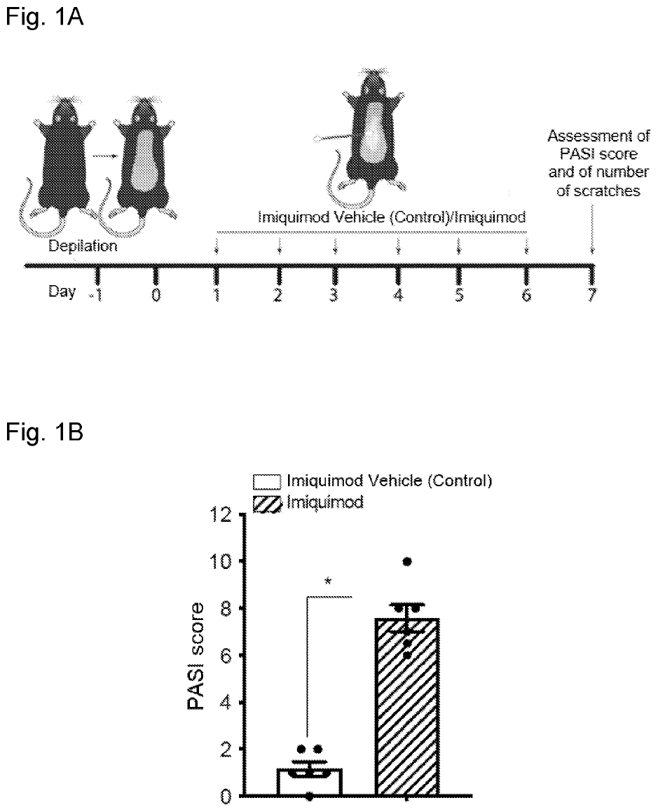

FIG. 1A schematically shows the treatment schedule of mice treated with imiquimod or imiquimod vehicle (referred to as control mice).

As it is possible to observe in FIG. 1B, the treatment with imiquimod cream has caused psoriatic lesions in the mouse. In FIG. 1B, it is possible to observe the PASI (Psoriasis Area Severity Index) score detected for control mice and imiquimod-treated mice. Data are shown as mean±SEM. * p<0.05 vs imiquimod vehicle (control); statistical analysis by Student's t-test.

The PASI index is a dermatological index introduced in the late 1970s to monitor the efficacy of treatments used for the treatment of psoriasis. For the assessment of the PASI score, some parameters are assessed and given a score, varying from 0 (no psoriasis) to 72 (maximum value, severe psoriasis). In the present experiment, the following parameters were assessed for the calculation of the PASI: appearance of erythema, scale formation and thickening of the skin. Each of these parameters was independently assessed on a scale of 0 to 4:0-none, 1-light, 2-moderate, 3-significant, 4-maximum. The cumulative score (erythema plus scaling plus thickening) was used to measure the severity of inflammation (0-12 scale). The score for each mouse was averaged. In FIG. 1B, it is possible to observe that the cumulative PASI score of the imiquimod-treated mice is 7.58±1.43 on a scale of 0-12. Therefore, it is reasonable to conclude that imiquimod treatment produced inflammation and typical psoriatic lesions. These mice are therefore good models for the study of psoriasis and of treatments useful to cure said disease.

### Example 2-Use of an Oligonucleotide Having Sequence Entirely Complementary to Murine miR-203b-3p microRNA

In particular, the Applicant has found that an oligonucleotide having sequence (SEQ: ID: NO. 2) complementary to the sequence of the murine miR-203b-3p microRNA (SEQ: ID: NO. 1) is capable of pairing with the entire sequence of the murine miR-203b-3p microRNA, inhibiting its action and blocking the final onset of itching.

The Sequence of the Murine miR-203b-3p microRNA Indicated with the Identifier (SEQ: ID: NO. 1) is Shown Hereinafter:

Sequence of Murine miR-203b-3p microRNA (SEQ: ID: NO. 1)

(SEQ: ID: NO. 1)
5′-UUG AAC UGU CAA GAA CCA CUG G-3′

The sequence of the oligonucleotide complementary to the murine miR-203b-3p microRNA (SEQ: ID: NO. 1) indicated with the identifier (SEQ: ID: NO. 2) is shown hereinafter:

Sequence of the Oligonucleotide Complementary to the Murine miR-203b-3p microRNA (SEQ: ID: NO. 2)

(SEQ: ID: NO. 2)
5′-CCA GUG GUU CUU GAC AGU UCA A-3′

In particular, the sequence (SEQ: ID: NO. 2) is a sequence of 22 nucleotides in the mouse entirely complementary to the sequence of the murine miR-203b-3p microRNA (SEQ: ID: NO. 1).

Said oligonucleotide (SEQ: ID: NO. 2) was used to test the inhibition efficacy of the murine miR-203b-3p microRNA and to assess the effect of reduction of the psoriasis-induced itching in murine models of psoriasis, so that these results could be translated to humans. The (SEQ: ID: NO. 2) is the sequence entirely complementary to the murine miR-203b-3p microRNA (SEQ: ID: NO. 1). The counterpart of the murine sequence (SEQ: ID: NO. 2) sequence in humans, i.e. the sequence entirely complementary to the human miR-203b-3p microRNA is the oligonucleotide having sequence (SEQ: ID: NO: 4).

In particular, C57BL/6J mice were treated with imiquimod (n=12) and control mice were treated with imiquimod vehicle (vaseline cream) (n=12). The mice treated with either imiquimod or imiquimod vehicle were divided into two further experimental groups of which 6 mice treated with imiquimod and 6 mice treated with imiquimod vehicle were treated with the sequence complementary to murine miR-203b-3p microRNA (Integrated DNA Technologies IDT, Tema Ricerche) administered intradermally (1 nmol, 10 μl) into the nape of the mouse every day for 6 consecutive days after imiquimod or imiquimod vehicle administration.

In addition, 6 mice treated with imiquimod and 6 mice treated with imiquimod vehicle were treated with the inhibitor vehicle (the solution in which the complementary sequence was diluted, represented by a 0.9% solution of sodium chloride, NaCl).

Treatments with the complementary sequence (SEQ: ID: NO. 2) or with the vehicle were carried out once daily from day 1 to day 6 of treatment with imiquimod or imiquimod vehicle.

On day 7, after the administration of imiquimod cream (5%) or control cream and treatment with the sequence complementary to murine miR-203b-3p microRNA (SEQ: ID: NO. 2) defined inhibitor, or with the inhibitor vehicle, the animals were placed in a 30 cm×30 cm arena and a 1-hour video was recorded.

The video was subsequently viewed by an operator who counted the number of times the animal scratched with its hind paw the area where imiquimod cream (5%) or control cream was applied after the treatment with the sequence complementary to murine miR-203b-3p microRNA (SEQ: ID: NO. 2) defined inhibitor, or with the inhibitor vehicle.

The measurement of the number of times the animal scratched itself with its hind paw the area of application of imiquimod cream (5%) or the control cream after the treatment with the sequence complementary to murine miR-203b-3p microRNA (SEQ: ID: NO. 2) or with its vehicle was defined as the number of scratches and is an index of the presence of itching.

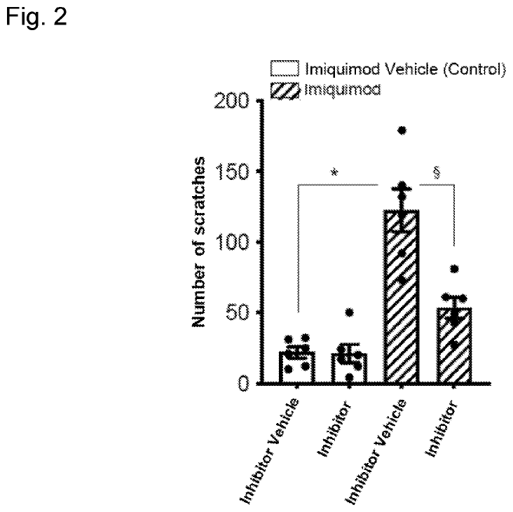

In the abscissa of FIG. 2, the term “inhibitor” indicates the treatment carried out with the oligonucleotide (SEQ: ID: NO. 2) complementary to the murine miR-203b-3p microRNA and the term “inhibitor vehicle” indicates the treatment carried out with the solution wherein the oligonucleotide complementary to the murine miR-203b-3p microRNA was diluted, represented by a 0.9% solution of sodium chloride, NaCl).

As it is possible to observe in FIG. 2, the treatment with imiquimod (5%) induced a significant increase in the number of scratches with respect to animals treated with control cream. The treatment with the sequence complementary to the murine miR-203b-3p microRNA was capable of reducing in mice treated with imiquimod (5%) (psoriasis model mice) the number of scratches in the area where imiquimod cream was applied (5%). This experiment shows that a sequence entirely complementary to the murine miR-203b-3p microRNA is capable of reducing the itching in a murine model of psoriasis induced by imiquimod (5%).

Data are shown as mean±SEM. * p<0.05 vs imiquimod vehicle (control); § p<0.05 vs imiquimod/inhibitor vehicle. Statistical analysis by one-way analysis of variance (ANOVA) and Bonferroni test.

### Example 3-Use of an Oligonucleotide Having Sequence Entirely Complementary to the Central Portion of Murine miR-203b-3p microRNA

In order to determine the efficacy of the central sequence 5′-UUCUUAAC-3′ of the antisense oligonucleotide for use in humans, studies were performed on mice, in particular by testing an oligonucleotide complementary to the central portion of the murine miR-203b-3p microRNA. Said oligonucleotide was used to assess the effect of reduction of psoriasis-induced itching in murine models of psoriasis, so that these results could be translated to humans.

An antisense oligonucleotide having a

5′-UUCUUGAC-3′

sequence complementary to the central portion (CORE sequence)

5′-GUCAAGAA-3′

of the multiple miR-203b-3p microRNA was obtained. In particular, the antisense oligonucleotide having sequence 5′-UUCUUGAC-3′ is an 8-nucleotide sequence in the mouse that is entirely complementary to the CORE sequence of the murine miR-203b-3p microRNA.

The antisense oligonucleotide used in the present experiment is the sequence entirely complementary to the central portion, or CORE sequence, of the murine miR-203b-3p microRNA sequence (SEQ: ID: NO. 1) shown in Example 2.

The sequence 5′-UUCUUGAC-3′ is a central sequence, or CORE sequence inhibitor, of the oligonucleotide complementary to the central sequence of the murine miR-203b-3p microRNA (SEQ: ID: NO. 2) shown in Example 2.

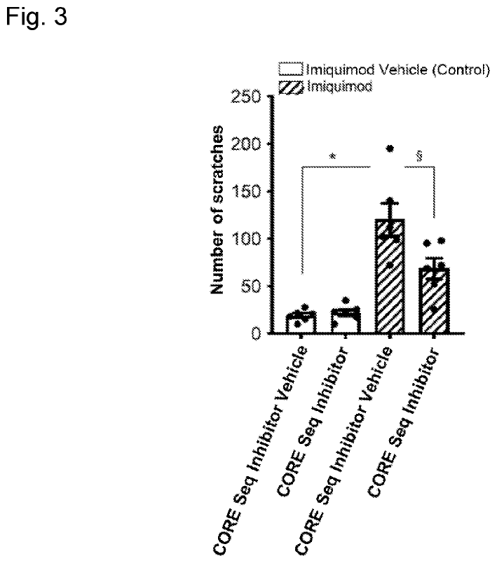

For this experiment, C57BL/6J mice were treated with imiquimod (n=12) and control mice were treated with imiquimod vehicle (vaseline cream) (n=12). The mice treated with imiquimod or with imiquimod vehicle were divided into two further experimental groups of which 6 mice treated with imiquimod and 6 mice treated with imiquimod vehicle were treated with the antisense oligonucleotide having the sequence 5′-UUCUUGAC-3′ complementary to the central portion, or CORE sequence, of the murine miR-203b-3p microRNA (Integrated DNA Technologies IDT, Tema Ricerche) (referred to as “CORE seq inhibitor” in FIG. 3) administered intradermally (1 nmol, 10 μl) into the nape of the mouse every day for 6 consecutive days after the administration of imiquimod (5%) or imiquimod vehicle.

Furthermore, 6 mice treated with imiquimod (5%) and 6 mice treated with imiquimod vehicle were treated with the antisense oligonucleotide vehicle, i.e. the solution in which the sequence complementary to the central sequence, or CORE sequence, represented by a 0.9% solution of sodium chloride, NaCl (referred to as “CORE seq inhibitor vehicle” in FIG. 3) was diluted.

Treatments with the antisense oligonucleotide with the vehicle were performed once daily from day 1 to day 6 of treatment with imiquimod (5%) or imiquimod vehicle.

On day 7, after the administration of imiquimod cream (5%) or control cream and treatment with the oligonucleotide having a sequence complementary to the central portion of the murine miR-203b-3p microRNA, or with vehicle, the animals were placed in a 30 cm×30 cm arena and a 1-hour video was recorded.

The video was then viewed by an operator who counted the number of times the animal scratched with its hind paw in the area where imiquimod cream (5%) or control cream had been applied after the treatment with antisense oligonucleotide as above defined or with vehicle.

The measurement of the number of times the animal scratched itself is an index of the presence of itching and has been defined as the number of scratches in FIG. 3.

As it is possible to observe in FIG. 3, the treatment with imiquimod (5%) induces a significant increase in the number of scratches with respect to animals treated with control cream. The treatment with the antisense oligonucleotide complementary to the central portion of the murine miR-203b-3p microRNA (shown in FIG. 3 as “CORE Seq Inhibitor”) was capable of reducing in mice treated with imiquimod (5%) (psoriasis model mice) the number of scratches in the area where imiquimod (5%) cream was applied. This experiment shows that an oligonucleotide having a sequence entirely complementary to the central portion of the murine miR-203b-3p microRNA is capable of reducing the itching in a murine model of psoriasis induced by imiquimod (5%).

Data are shown as mean±SEM. * p<0.05 vs imiquimod vehicle (control); $p<0.05 vs imiquimod/CORE Seq inhibitor vehicle. Statistical analysis by one-way analysis of variance (ANOVA) and Bonferroni test.

1. A method for the treatment of psoriasis-induced itching, comprising administering to a subject in need thereof an effective amount of an antisense oligonucleotide complementary to human miR-203b-3p microRNA (SEQ: ID: NO. 3), said oligonucleotide having sequence (SEQ: ID: NO. 4) or sequence with an identity percentage equal to at least 70% with respect to said sequence (SEQ: ID: NO. 4);

2. A method according to claim 1, wherein said antisense oligonucleotide comprises a central sequence of 8 nucleotides 5′-UUCUUAAC-3′.

3. A method according to claim 2, wherein the first uracil (U) of the central sequence UUCUUAAC corresponds to the base in the ninth nucleotide in the oligonucleotide and the last cytosine (C) of the central sequence corresponds to the base in the sixteenth nucleotide in the oligonucleotide.

4. A method according to claim 2, wherein said central sequence 5′-UUCUUAAC-3′ within said sequence (SEQ: ID: NO. 4) binds the central portion 5′-GUUAAGAA-3′ of the sequence (SEQ: ID: NO. 3) of human miR-203b-3p microRNA.

5. A method according to claim 1, wherein said antisense oligonucleotide is in the form of antagomir of the human miR-203b-3p microRNA (SEQ: ID: NO. 3).

6. A method according to claim 1, wherein said antisense oligonucleotide with an identity percentage equal to at least 70% with respect to said sequence (SEQ: ID: NO. 4) is selected from the group that comprises the following sequences:
(SEQ: ID: NO. 5)
5′-ACA ACU UGU UCU UAA CAU GUC CA-3′
(SEQ: ID: NO. 6)
5′-UCA CGU ACU UCU UAA CAU UUA CA-3′
(SEQ: ID: NO. 7)
5′-AUG ACU GGU UCU UAA CAG UAG UA-3′
(SEQ: ID: NO. 8)
5′-AGC UCA GGU UCU UAA CAG UUC AA-3′
(SEQ: ID: NO. 9)
5′-UUA AGU GGU UCU UAA CAG CUA CA-3′
(SEQ: ID: NO. 10)
5′-UGC UGA GGU UCU UAA CAG ACC AA-3′
(SEQ: ID: NO. 11)
5′-UCA AAC GGU UCU UAA CAG UUC AA-3′
(SEQ: ID: NO. 12)
5′-UCC CGU GGU UCU UAA CAG CUC GA-3′
(SEQ: ID: NO. 13)
5′-UGC AGC GGU UCU UAA CAG UUA AA-3′.

7. A method according to claim 1, wherein said psoriasis is selected from plaque psoriasis, pustular psoriasis, guttate psoriasis, inverse psoriasis and erythrodermic psoriasis.

8. A method according to claim 7, wherein said psoriasis is plaque psoriasis.

9. A method for the treatment of psoriasis-induced itching, comprising administering to a subject in need thereof an effective amount of a phospholipid vesicle that comprises at least an antisense oligonucleotide complementary to human miR-203b-3p microRNA (SEQ: ID: NO. 3), said oligonucleotide having sequence (SEQ: ID: NO. 4) or sequence with an identity percentage equal to at least 70% with respect to said sequence (SEQ: ID: NO. 4).

10. A method according to claim 9, wherein said phospholipid vesicle comprises at least an oligonucleotide selected from the group of oligonucleotides having sequence (SEQ: ID: NO. 5), (SEQ: ID: NO. 6), (SEQ: ID: NO. 7), (SEQ: ID: NO. 8), (SEQ: ID: NO. 9), (SEQ: ID: NO. 10), (SEQ: ID: NO. 11), (SEQ: ID: NO. 12) and (SEQ: ID: NO. 13).

11. A method according to claim 9, wherein said phospholipid vesicle comprises the oligonucleotide having sequence (SEQ: ID: NO. 4) and at least a further oligonucleotide selected from the group of oligonucleotides having sequence (SEQ: ID: NO. 5), (SEQ: ID: NO. 6), (SEQ: ID: NO. 7), (SEQ: ID: NO. 8), (SEQ: ID: NO. 9), (SEQ: ID: NO. 10), (SEQ: ID: NO. 11), (SEQ: ID: NO. 12) and (SEQ: ID: NO. 13).

12. A method according to claim 9, wherein said phospholipid vesicle is selected from the group consisting of a liposome, a phytosome, a micelle, and mixtures thereof.

13. A method according to claim 12, wherein said phospholipid vesicle is in the form of a liposome.

14. A method according to claim 9, wherein said psoriasis is selected from the group consisting of plaque psoriasis, pustular psoriasis, guttate psoriasis, inverse psoriasis and erythrodermic psoriasis.

15. A method according to claim 14, wherein said psoriasis is plaque psoriasis.

16. A method for the treatment of psoriasis-induced itching, comprising administering to a subject in need thereof an effective amount of a pharmaceutical composition which comprises at least a phospholipid vesicle and one or more pharmaceutically suitable or pharmaceutically acceptable excipient(s)
wherein said phospholipid vesicle comprises at least an antisense oligonucleotide complementary to human miR-203b-3p microRNA (SEQ: ID: NO. 3), said oligonucleotide having sequence (SEQ: ID: NO. 4) or sequence with an identity percentage equal to at least 70% with respect to said sequence (SEQ: ID: NO. 4).

17. A method according to claim 16, wherein said pharmaceutical composition is in the form of cream, powder, or ointment.

18. A method according to claim 17, wherein said pharmaceutical composition is in the form of cream for topical use.

19. A method according to claim 16, wherein said pharmaceutical composition is in a form suitable for application by means of a medicated patch.

20. A method according to claim 16, wherein said psoriasis is selected from the group consisting of plaque psoriasis, pustular psoriasis, guttate psoriasis, inverse psoriasis and erythrodermic psoriasis.

21. A method according to claim 20, wherein said psoriasis is plaque psoriasis.

22. A method of treatment of psoriasis-induced itching comprising the topical application on at least one psoriatic lesion of a subject suffering from psoriasis of a therapeutically effective amount of a composition which comprises at least a phospholipid vesicle and one or more pharmaceutically suitable or pharmaceutically acceptable excipient(s)
wherein said phospholipid vesicle comprises at least an antisense oligonucleotide complementary to human miR-203b-3p microRNA (SEQ: ID: NO. 3), said oligonucleotide having sequence (SEQ: ID: NO. 4) or sequence with an identity percentage equal to at least 70% with respect to said sequence (SEQ: ID: NO. 4).

23. A method of treatment according to claim 22, further comprising the administration of at least one drug for the treatment of psoriasis.

24. A method of treatment according to claim 23, wherein said at least one drug for the treatment of psoriasis is selected from the group consisting of a corticosteroid, a topical analogue of D3 vitamin, a calcineurin inhibitor, and tazarotene.

25. A method of treatment according to claim 23, wherein said at least one drug for the treatment of psoriasis is administered before, after, or concurrently with the topical treatment with said composition.
